(19) United States
(12) Patent Application Publication
(10) Pub. No.: US 2021/0393310 A1
(43) Pub. Date: Dec. 23, 2021

(54) METHOD FOR CONTROLLING A MEDICAL DEVICE AND A MEDICAL DEVICE IMPLEMENTING THE SAME

(71) Applicant: OLYMPUS CORPORATION, Tokyo (JP)

(72) Inventor: Kotaro NAKAMURA, Sagamihara-shi (JP)

(73) Assignee: OLYMPUS CORPORATION, Tokyo (JP)

(21) Appl. No.: 17/347,827

(22) Filed: Jun. 15, 2021

Related U.S. Application Data
(60) Provisional application No. 63/042,594, filed on Jun. 23, 2020.

(51) Int. Cl. A61B 18/08; A61B 18/12

(52) U.S. Cl. CPC A61B 18/082; A61B 2018/00714; A61B 18/1206

(57) ABSTRACT

A method for controlling a temperature at an end-effector of an instrument connected with a controller includes estimating a residual energy associated with a prior application of base energy to the end-effector based on a first set of parameters. An amount of electric power that is converted to heat at the end-effector is estimated based on the first set of parameters. A current temperature at the end-effector is estimated based on: (i) the residual energy, (ii) the amount of electric power provided to the end-effector, and (iii) a time for which the electric power is provided. The electric power provided to the instrument is controlled to maintain the current temperature at the end-effector within a predetermined range.

### RELATED APPLICATION DATA

This application is based on and claims priority under 35 U.S.C. § 119 to U.S. Provisional Application No. 63/042,594, filed Jun. 23, 2020, the entire contents of which are incorporated herein by reference.

### TECHNICAL FIELD

The systems, devices and methods disclosed herein are directed to medical devices, and in particular to medical devices including an end-effector used for heating tissue.

### BACKGROUND

Many medical procedures require heating a body tissue. For example, there may be a need for cauterizing a blood vessel or shearing a piece of tissue in certain procedures. In other medical procedures, there may be a need for welding two pieces of tissue together.

Various medical devices may be used for heating body tissue and these medical devices may include different end-effectors for heating the body tissue. Depending on the body tissue and the medical procedure, heating can be accomplished using, for example, heat energy, ultrasound energy, electric energy (e.g., high frequency current), light energy, or any other suitable forms of energy that could be transmitted through the end-effectors. It is important to maintain the temperature of the body tissue at a level that is safe for surrounding body tissue, while also being effective for performance of the procedure being performed. However, achieving this balance can be difficult because estimating the temperature at the end-effector can be challenging.

### SUMMARY

In one aspect of the present disclosure, a method for controlling a temperature of an end-effector of a medical device includes determining a base energy applied to the end-effector based on the electric power provided to the medical device and estimating a residual energy associated with a prior application of base energy to the end-effector based on a first set of parameters specific to the end-effector. An amount of electric power converted to heat at the end-effector based on the base energy and the residual energy is computed. A temperature associated with the residual energy is estimated. A change in temperature associated with the change in energy of the end-effector based on the first set of parameters specific to the end-effector is estimated. A current temperature at the end-effector based on the temperature associated with the residual energy and the temperature associated with the change in energy is estimated. The electric power provided to the medical device is controlled so as to maintain the current temperature of the end-effector within a predetermined range.

In another aspect of the present disclosure, a method for controlling a temperature of an end-effector of the medical device includes determining a base energy applied to the end-effector based on the electric power provided to the medical device and estimating a residual energy associated with a prior application of base energy to the end-effector based on a first set of parameters specific to the end-effector. A current temperature at the end-effector is estimated based on: (i) the residual energy, (ii) the amount of electric power converted to heat at the end-effector, and (iii) a time for which the heat is supplied. The electric power provided to the medical device is controlled so as to maintain the current temperature of the end-effector within a predetermined range.

In yet another aspect of the present disclosure, medical device for applying heat to a tissue includes an end-effector configured to contact the tissue and to transmit heat to the contacted tissue, a power source configured to provide electric power to the medical device, and a processor. The processor is configured to compute an amount of electric power converted to heat at the end-effector and estimate of the current temperature at the end-effector based on: (i) a first characteristic of the end-effector, (ii) the amount of electric power converted to heat at the end-effector, and (iii) a time for which the heat is supplied. The power provided to the medical device is controlled so as to maintain the current temperature of the end-effector within a predetermined range.

Additional features and advantages will be set forth in the description that follows, and in part will be apparent from the description, or may be learned by practice of the invention. The objectives and other advantages of the disclosed input device will be realized and attained by the structure particularly pointed out in the written description and claims thereof as well as the appended drawings.

### BRIEF DESCRIPTION OF THE DRAWINGS

The disclosed aspects will hereinafter be described in conjunction with the appended drawings, provided to illustrate and not to limit the disclosed aspects, wherein like designations denote like elements.

FIG. 1 shows a schematic of a medical device for heating a tissue, in accordance with an embodiment of the present disclosure.

FIG. 2 shows a schematic of a controller in accordance with an embodiment of the present disclosure.

FIG. 3 shows a flow chart for a process of maintaining a temperature at an end-effector of a medical device for heating a tissue, in accordance with an embodiment of the present disclosure.

FIGS. 4A and 4B are graphs of power (W) and estimated temperature (degrees C.) as a function of time (seconds) related to aspects of the disclosed method for controlling a temperature at an end-effector.

### DETAILED DESCRIPTION

Medical devices that can provide heat to a body tissue are used in various surgical procedures, for example, to cauterize the blood vessel, or to weld or seal tissues or lumens. One of the common types of devices includes an end-effector that can produce ultrasound vibration at the site of the end-effector. However, the ultrasound vibration makes it difficult to include a temperature sensor at the end-effector. This poses a challenge in accurately estimating the temperature at the site of the end-effector.

Without an accurate estimation of the temperature at the end-effector, it is difficult to control the energy output at the site of the end-effector causing in undesirable rise in temperature or insufficient heating at the site of the end-effector. While undesirable rise in temperature can cause damage to the tissue, insufficient heating may result in an incomplete procedure. In both situations patient safety may be compromised.

Accordingly, one aspect of the present disclosure describes a method for accurately estimating the temperature at the site of the end-effector. The present disclosure further describes control of the medical device based on the estimated temperature at the site of the end-effector. In another aspect, present disclosure further relates to a medical device, per se, which operates based on the method for accurately estimating the temperature at the site of the end-effector.

### A. Medical Device for Heating Tissue

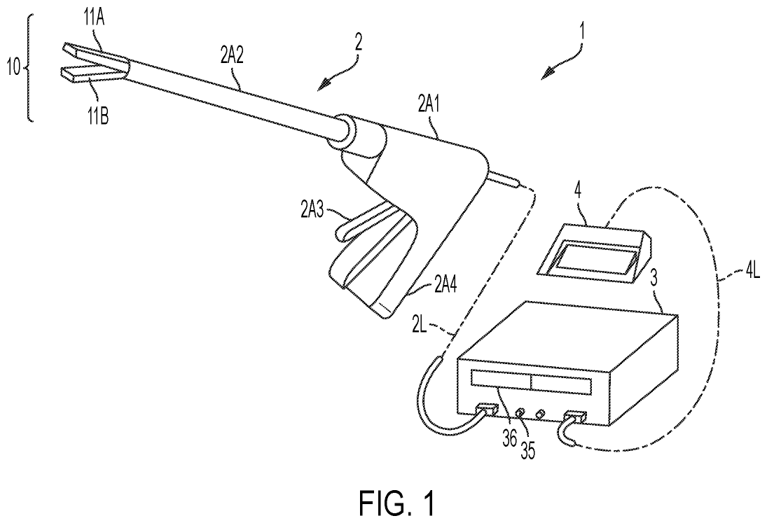

FIG. 1 shows a schematic of a medical device for heating a tissue, in accordance with an embodiment of the present disclosure. As shown in FIG. 1, the medical device 1 for heating a tissue is provided with an instrument 2, a controller 3 including an input controller 4 having a processor and an actuation switch. The instrument 2 may be, for example, a surgical operation energy inosculation apparatus used for welding living tissue, such as in an abdominal cavity through an abdominal wall, or incising, such as in an open surgery procedure or laparoscopy.

The treatment instrument 2 has a grip 2A1, a shaft 2A2, and a treatment section constituted by an end-effector 10 such as, for example, an openable or pivoting pair of grasping sections 11 (including a first grasping section 11A and a second grasping section 11B) for grasping living tissue LT to perform treatment. The grasping sections 11 as whole are also referred to herein as the “treatment portion” or the “treatment section” of the medical instrument. Note that, hereinafter, at time of mentioning each of components having a same function and having reference numerals with A and B attached to ends of the reference numerals, respectively, the symbol A or B may be omitted. For example, each of the first grasping section 11A and the second grasping section 11B may be referred to as the grasping section 11.

The grip 2A1 is connected to the controller 3 via a cable 2L. The grip 2A1 has an opening/closing actuator 2A3, such as a trigger, for a surgeon to operate opening and closing of the treatment section is in such a shape that the surgeon can easily clasp, for example, in a substantially L shape. The opening/closing actuator 2A3 is arranged at one end of the grip 2A1 and is integrated with the treatment section to transmit operation of the opening/closing actuator 2A3 to the treatment section. On the other side of the grip 2A1, a grasping portion 2A4 is provided for a clinician to grasp when operating the instrument 2.

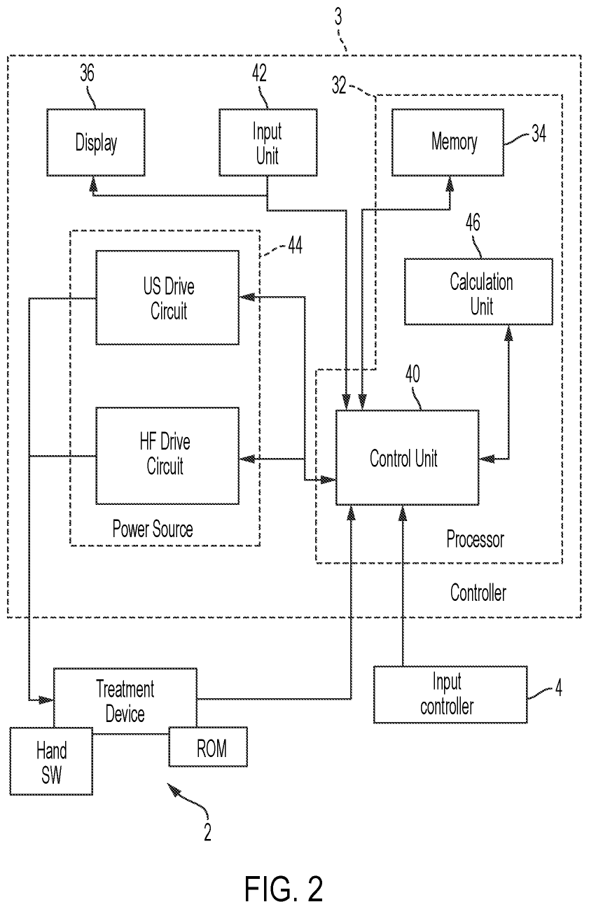

FIG. 2 shows a schematic of a controller in accordance with an embodiment of the present disclosure. The controller 3 may include a processor 32, a display 36, an input unit 42, and a power source 44.

The processor 32 may include a memory 34, a calculation unit 46 and a control unit 40. The calculation unit 46 and the control unit 40 are formed of an integrated circuit including a CPU (Central Processing Unit), an ASIC (Application Specific Integrated Circuit) or an FPGA (Field Programmable Gate Array). The calculation unit 46 and the control unit 40 may be formed a single integrated circuit, or may be formed of a plurality of integrated circuits.

In some embodiments, various parameters used for estimating the current temperature such as, for example, the specific heat capacity of the treatment portion, the thermal resistance of the treatment portion, the thermal conductivity of the treatment portion, or a contact area between the body tissue and the treatment portion, any of which may be stored in memory 34, e.g., in a look-up table stored in the memory 34. The look-up table may include the values of the corresponding parameters for different treatment portions. For example, the look-up table may include the parameters for muscle tissue, adipose tissue, blood vessels, intestinal wall, or other tissue types. In such embodiments, estimating the current temperature may include determining the type of tissue on which the procedure is being performed, and determining the corresponding parameter values in the look-up table for estimating the current temperature. Alternatively, the procedure name may be used as the basis for entry into the look-up table. The calculation unit 46 is configured to compute the estimated current temperature as well as other parameters that are needed for computing the estimated current temperature. The control unit 40 is configured to control the power source 44 and the display 36 based on the commands provided by the processor 32 using the parameters computed by the calculation unit 46.

In some embodiments, the data related to the various parameters is a value of the contact area calculated using size and shape of the end-effector, in particular, the treatment portion of the end-effector, and how the end-effector is used (e.g., related to the procedure) and this data is stored in the memory in advance. In addition, as the contact area changes as the end-effector grasps and regrasps tissue during the medical procedure, the various parameters can also correspondingly change during the medical procedure, and having the data for the various parameters readily available in memory allows for dynamic updating during the medical procedure.

The display section 36 which displays treatment conditions and the like, and a setting operation section 35 for the surgeon to set the treatment conditions and the like on a front panel. In some embodiments, a foot switch 4 may be connected to the controller 3 via a cable 4L. By the clinician may turn the power output from the controller 3 to the treatment instrument 2 ON or OFF by an input controller 4, such as by pressing a pedal of a foot switch of the input controller 4. The foot switch is not an essential component and any input controller can be utilized, such as a switch or the like which the clinician operates by hand or other alternative input controller.

In some embodiments, the controller 3 includes a processor 32 which controls the application of power to the instrument 2 via the power source 44 so as to maintain a temperature at the end-effector 10 in a safe and effective range. In some embodiments, the processor 32 may override the input provided by the clinician (e.g., through the input controller 4) for turning the power to the instrument 2 ON or OFF.

To control the power input to the instrument 2, the processor 32 may estimate a temperature at the end-effector 10 using the methods described herein, and determine whether the temperature is within a predetermined range, at for example, the calculation unit 46. The predetermined range of temperature can be set by the clinician, for example, before beginning the procedure, using the setting operation section 35, which is input to the processor via the input unit 42.

Once the predetermined range of temperature is set, the calculation unit 46 may estimate the temperature at the end-effector 10. If the temperature at the end-effector 10 is greater than the upper limit of the predetermined range, the processor 32 may cause the control unit 40 to cut off power provided to the instrument 2 by the power source 44 regardless of whether the clinician continues to input control signals for the electrical power to remain ON.

In some embodiments, the calculation unit 46 may estimate an amount of time for which the power to instrument 2 is to be cut off such that temperature at the end-effector 10 remains within the predetermined range. In some embodiments, the amount of time for which the power to instrument 2 is to be cut off is determined based on the estimated current temperature, the upper limit of temperature, and a pre-calculated rate of cooling. In some embodiments, the control unit 40 may cause the power source 44 to decrease the power delivered to the instrument 2 at a certain rate depending on the amount by which the estimated temperature and end-effector 10 exceeds the upper limit of the predetermined range.

On the other hand, if the temperature at the end-effector 10 is lower than the lower limit of the predetermined range, the control unit 40 may cause the power source 44 to continue to supply power to the instrument 2 even if the clinician has caused the electrical power to be turned OFF.

In some embodiments, the controller 3 may also include an alarm which is coupled to the processor 32. The alarm may be audio (e.g., a speaker (not shown)), visual (e.g., display 36), audiovisual, haptic or a combination thereof. In such embodiments, when the processor determines that the temperature at the end-effector 10 is greater than the upper limit of the predetermined range, the processor 32 via the control unit 40 may cause the alarm to be turned on so as to provide a warning signal to the clinician. In some embodiments, the processor may cause the intensity of the warning signal to increase as the temperature at the end-effector 10 continues to increase above the upper limit of the predetermined range and/or the intensity can be related to the rate of change of the temperature.

In some embodiments, the control unit 40 may cause the display section 36 to display the estimated current temperature. For example, the estimated current temperature may be displayed as a function of time on a graph. Thus, a clinician may be able to visually estimate whether the current temperature has increased above a first threshold temperature that may, or be on a trend to, compromise patient safety. Similarly, the clinician may be able to visually estimate whether the current temperature has decreased below a second threshold temperature that may, or be on a trend to, be ineffective for the procedure being performed.

In some embodiments, the control unit 40 may be configured to cause the instrument 2 to increase or decrease a grasping force with which the instrument 2 or the end-effector 10 thereof grasps the portion of tissue on which the procedure is being performed.

### B. Estimating the Temperature of the End-Effector

The discussion that follows describes various methods for estimating the temperature of the end-effector 10 of the medical device 1.

Regardless of the mechanism by which the electrical power provided to the medical device 1 is converted to heat, the amount of heat produced at the end-effector is proportional to the product of the applied electrical power and the time for which the electrical power is applied. Thus, it is possible to estimate heat produced at the end-effector based on the amount of electrical power provided to the medical device 1, amount of electrical power consumed by the medical device 1, an amount of electrical power applied to the end-effector 10, an amount of electrical power consumed by the end-effector 10, or a combination thereof.

In one embodiment, heat (Q) produced by the end-effector can be calculated as:

Q=f(W·t) Equation (1)

where f is a constant specific to the medical device, W is the electrical power input to the medical device, and t is the sampling time. The constant f is indicative of the efficiency with which the medical device converts the inputted electrical energy into heat. The temperature of at the end-effector can then be calculated as:

$${T}_{C} = {T}_{prev} + \frac{fWt}{m c} \qquad Equation ( 2 )$$

where T_{c} is the current temperature, Tₚᵣₑᵥ is the temperature before the beginning of the sampling time, m is a mass of the portion of the tissue surrounding the end-effector and which receives the treatment applied by the end-effector (e.g. by the treatment portion of the end-effector), and c is the specific heat capacity of the treatment portion.

The mass m of the treatment portion is determined based on the size and shape of the end-effector as well as the type of tissue on which the procedure is being performed. For example, by the shape and size of the end effector, each end effector is operative on a specific known volume of tissue from which a mass m of the treatment portion can then be determined. The specific heat capacity c of the treatment portion depends on the type of the tissue on which the procedure(s) are being performed.

The temperature Tₚᵣₑᵥ before the beginning of the sampling time is interchangeably referred to herein as the residual temperature, and depends on the power input to the medical device before the sampling time. For example, prior to a first application of power input to the medical device, the temperature of the end effector can be at a temperature of its surroundings, such as the room temperature, a temperature of an autoclave, or the temperature of the patient. Subsequent to completing a first application of power input to the medical device, the medical device will be at some residual temperature that is dependent on the final temperature when power input was being applied and the rate of decay (or cooling) of the end effector. The residual temperature at the end-effector may be calculated using the equation 2. In other words, the estimated current temperature is determined iteratively.

### 1. Embodiment 1: Subtracting the Amount of Power Input Prior to the Sampling Time from the Power Input During the Sampling Time

The amount of heat generated by the end-effector is based on the power input during a given sampling time. Thus, the estimated current temperature at the end-effector may be more accurately calculated by subtracting the amount of power input prior to the sampling time. Those of ordinary skill in the art will appreciate that at least some of the heat generated by the end-effector is dissipated by conduction through the tissue on which the procedure is being performed. Thus, the current temperature of the treatment portion is the sum of the temperature of the treatment portion prior to the sampling time and the rise in the temperature at the end-effector because of the electrical power provided to or consumed by the medical device being converted to heat during the sampling time.

The change in temperature of the treatment portion during the sampling time is calculated as follows:

$$Δ T = ( \frac{fW - \frac{{T}_{prev}}{{R}_{th}}}{m \times c} ) / t \qquad Equation ( 3 )$$

where Rₜₕ is referred to as thermal resistance of the treatment portion, and is calculated as:

$${R}_{t h} = \frac{L}{k \times A} \qquad Equation ( 4 )$$

where k is the thermal conductivity of the treatment portion; A is the contact area between the treatment portion of the end-effector and the body tissue; and L is the thickness of the treatment portion.

The current temperature at the end-effector can then be estimated as:

T=ΔT+Tₚᵣₑᵥ Equation (5)

In some embodiments, the clinician may initiate the heating of the end-effector after inserting the instrument such that the end-effector is positioned at the treatment portion. As discussed herein, the typical treatment portion is inside the human body. Thus, in such embodiments, the initial temperature of the end-effector at the treatment portion is assumed to be T₀=37° C. (approximately equal to the temperature inside the human body) and used as Tₚᵣₑᵥ for the first sampling period. The current temperature is then iteratively estimated for each successive sampling period, during the time of the procedure.

In some embodiments, the sampling time may be, for example, 0.1 s, 0.2 s, 0.3 s, 0.4 s, 0.5 s, 0.6 s, 0.7 s, 0.8 s, 0.9 s, 1.0 s, 1.5 s, 2.0 s, 2.5 s, 3 s, 5 s, 10 s, or any other value between any two of these values. Thus, in some embodiments, a clinician may obtain a desired accuracy of the estimated temperature by appropriately selecting the sampling time. In some embodiments, the sampling time is pre-selected based on the end-effector being used such that the accuracy of the estimated current temperature may be adjusted based on the particular procedure being performed. In some embodiments, the sampling time may be preset for the medical device when the medical device is manufactured, in which case the preset value may be programmed into the controller 3 by the user or can be communicated to the controller 3 by the medical device when the medical device is assembled to the controller 3.

In some embodiments, various parameters used for estimating the current temperature such as, for example, the specific heat capacity of the treatment portion, the thermal resistance of the treatment portion, the thermal conductivity of the treatment portion, or the contact area between the body tissue and the treatment portion, any of which may be provided in, e.g., a look-up table which may include the values of the corresponding parameters for different treatment portions. For example, the look-up table may include the parameters for muscle tissue, adipose tissue, blood vessels, intestinal wall, or other tissue types. In such embodiments, estimating the current temperature may include determining the type of tissue on which the procedure is being performed, and determining the corresponding parameter values in the look-up table for estimating the current temperature. Alternatively, the procedure name may be used as the basis for entry into the look-up table.

In some embodiments, the data related to the various parameters is a value of the contact area calculated using size and shape of the end-effector, in particular, the treatment portion of the end-effector, and how the end-effector is used (e.g., related to the procedure) and this data is stored in the memory in advance. In addition, as the contact area changes as the end-effector grasps and regrasps tissue during the medical procedure, the various parameters can also correspondingly change during the medical procedure, and having the data for the various parameters readily available in memory allows for dynamic updating during the medical procedure.

### 2. Embodiment 2: Subtracting Base Ultrasound Power Consumption from Input Power

In some embodiments, the medical device may include an end-effector that is configured to heat the surrounding tissue (i.e., the treatment portion) by application of ultrasound waves. Ultrasound waves typically refer to sound waves having a frequency of greater than 20 kHz, and up to several GHz. Vibrations from the ultrasound waves results in heating the tissue surrounding the end-effector of the medical device. Thus, for an end-effector configured to heat the treatment portion by application of ultrasound waves, Equation (4) can be modified as:

$$Δ T = ( \frac{( W - {W}_{U S} ) - \frac{k \times A \times {T}_{p r e v}}{L}}{m \times c} ) / t \qquad Equation ( 3 ’ )$$

where W is power input to the medical device during the procedure over the sampling time t and W_{US} is the power needed to produce ultrasound waves at the same frequency and intensity as being produced during the procedure assuming there was no load on the end-effector.

In some embodiments, the value of W_{US} can be obtained based on the physics of the end-effector (e.g., using the dimensions, material properties, and other physical characteristics of the end-effector). In some embodiments, the value of W_{US} can be determined using a look-up table that includes the input power needed to generate ultrasound waves of certain frequencies and intensities.

In some embodiments, the data related to the various parameters is a value of the contact area calculated using size and shape of the end-effector, in particular, the treatment portion of the end-effector, and how the end-effector is used (e.g., related to the procedure) and this data is stored in the memory in advance. In addition, as the contact area changes as the end-effector grasps and regrasps tissue during the medical procedure, the various parameters can also correspondingly change during the medical procedure, and having the data for the various parameters readily available in memory allows for dynamic updating during the medical procedure.

### 3. Embodiment 3: Factoring Change in Resonance Frequency of the Applied Ultrasound Waves

In medical devices where the end-effector is configured to heat the surrounding tissue by application of ultrasound waves, when performing the procedure there may be instances when the parts of the surrounding tissue may adhere to or coagulate around the end-effector. Such adhesion or coagulation may result in reduction in the resonance frequency of the end-effector because of the change in physical characteristics of the end-effector. The decrease in resonance frequency of the end-effector causes a reduction in kinetic energy of the end-effector, thereby reducing the energy consumption of the end-effector.

Thus, the estimate of the current temperature at the end-effector determined, e.g., in Embodiment 2 described herein, may be further improved in some instances by factoring the reduction in resonance frequency of the end-effector. This may be particularly applicable when the treatment portion interacts with tissue(s) that are susceptible to adhesion to the end-effector or coagulation around the end-effector.

In such instances, Equation (3′) is further modified as:

$$Δ T = ( \frac{( W - {W}_{U S} ) \times \frac{{f}_{r}}{{f}_{r 0}} - \frac{k \times A \times {T}_{p r e v}}{L}}{m \times c} ) / t \qquad Equation ( 3 ” )$$

where fᵣ is the current resonance frequency of the end-effector during the sampling period, and fᵣ₀ is the base resonance frequency of the end-effector.

It will be further appreciated that in instances where the end-effector is configured to grasp a portion of tissue when performing the procedure, the grasping force may affect the mode of vibration, and thus, may affect the estimate of the current temperature. However, it will be apparent to those of ordinary skill in the art that any change in resonance frequency or mode of vibration of the end-effector during the procedure is measurable and is factored into Equation (3″). Moreover, measuring the current resonance frequency during a procedure is well-understood in the art. For example, the initial resonant frequency of the device can be determined by the composition of the material generating the ultrasound (e.g., lead zirconate titanate, PZT) and the end effector. The resonance frequency may be, e.g., about 47 kHz in some embodiments. The resonance frequency during the treatment is dependent on the impedance of the end-effector, and thus, can be estimated by, e.g., impedance matching. At that time, the frequency with the lowest impedance is determined as the current resonance point for the purposes of Equation (3″).

Various parameters used in estimating the current temperature in accordance with the present disclosure, such as, for example, k, L, A, m, and c, are determined based on the particular end-effector, particular procedure being performed, and the particular tissue being treated. Additionally, features of the treatment portion (such as area, thickness, volume, etc.) and the contact area between the body tissue and the treatment portion are determined based on the physical characteristics of the end-effector (such as the size and shape). Parameters such as thermal conductivity and specific heat capacity for various materials for the treatment portion as well as for body tissues are well-known in the art and those of ordinary skill in the art will appreciate that appropriate modifications to those parameters may be needed based on the particular use case.

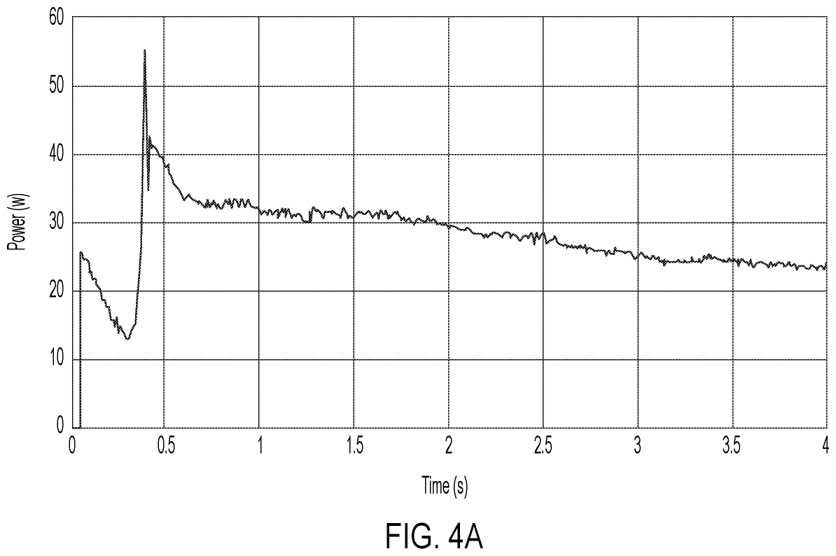

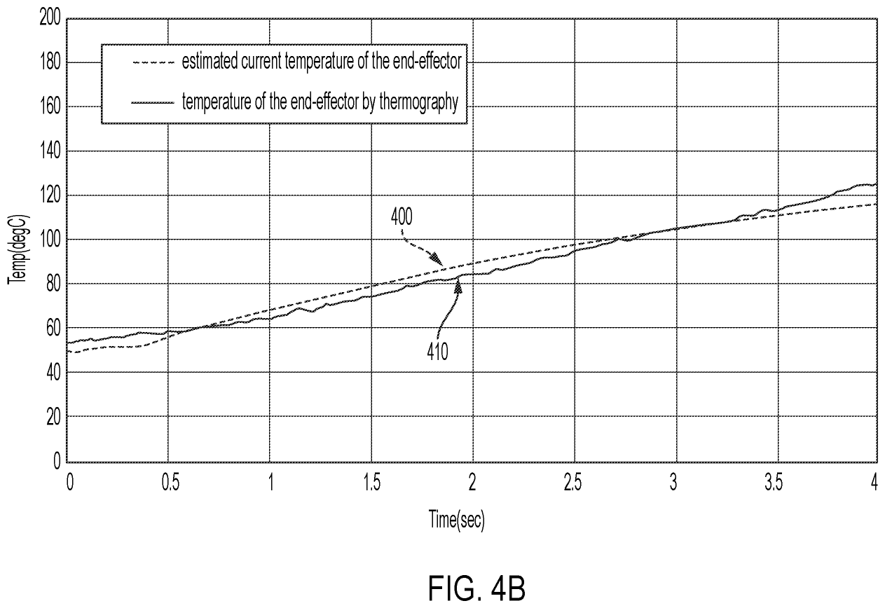

FIG. 4A shows an example of the measured value of power outputted by a generator connected to an end-effector. For this example, the parameters of the end-effector are: m=1.5 g, c=0.585 J/gK, k=7.5 W/mK, A=0.0000075 m², and L=0.0015 m. FIG. 4B shows a comparison between the estimated temperature of the end-effector calculated using Equations (5) and (3″) (see 400 in FIG. B) and the temperature of the end-effector determined by thermography (See 410 in FIG. 4B). As can be seen in FIG. 4B, the estimated temperature based on the method disclosed herein tracks closely with the temperature determined by thermography.

### 4. Embodiment 4: Regulating the Temperature at the End-Effector

Accurate estimation of the temperature at the end-effector is important for preventing over-heating the tissue on which the procedure is being performed. Thus, once the temperature at the end-effector is estimated, in some embodiments, the power input to the medical device may be regulated to ensure that the tissue is not over-heated, thereby enhancing patient safety.

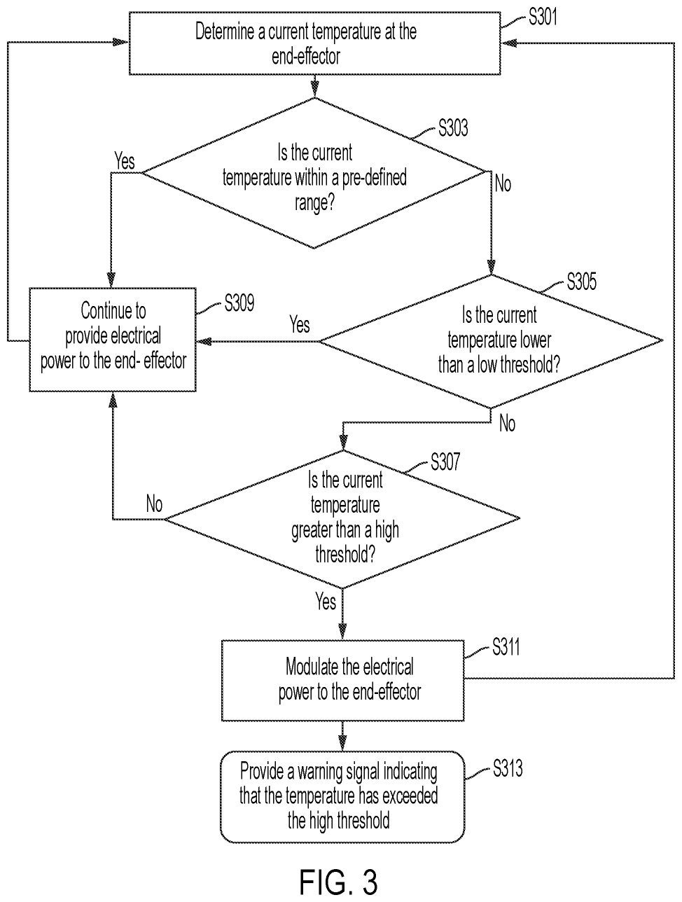

In one aspect, regulating the temperature includes maintaining the estimated current temperature at the end-effector within a predetermined range. FIG. 3 shows a flow chart for a process of maintaining a temperature at an end-effector of a medical device for heating a tissue, in accordance with an embodiment of the present disclosure.

In an example embodiment, a method of maintaining a temperature at an end-effector of a medical device includes at S301, determining a current temperature at the end-effector. Any one of the methods described herein may be used for estimating the current temperature for regulating the temperature at the end-effector.

The method further includes, at S303, determining whether the current temperature is within a pre-defined range. If the current temperature at the end-effector is determined to be within the pre-defined range, at S309, provision of electrical power to the end-effector is continued and the method reverts to S301, where the current temperature at the end-effector is determined again.

If it is determined at S303 that the current temperature at the end-effector is not within the pre-defined range, at S305, it is determined whether the current temperature at the end-effector is lower than a lower limit of the pre-defined range (also referred to herein as the low threshold or the third threshold). In such instances, the method moves to S309 where provision of electrical power to the end-effector is continued and the method reverts to S301, where the current temperature at the end-effector is determined again.

In some embodiments, at S309, the electrical power input to the medical device is increased. Thus, in some embodiments, a rate at which to increase the power input to the medical device is determined based on a rate of change in the estimated current temperature over a certain number of previous sampling cycles. For example, in some embodiments, the rate of change in the estimated current temperature is determined over 1, 2, 3, 4, 5, 6, 7, 8, 9, 10, 12, 14, 16, 18, 20, 25, 30 or any number between any two of these numbers, previous sampling cycles, and the power input to the medical device is increased or decreased based on the determined rate of change in the estimated current temperature and, optionally, also considering the selected temperature and the difference from the estimated current temperature and the selected temperature.

If it is determined at S305 that the current temperature at the end-effector is not lower than the low threshold, at S307, it is determined whether the current temperature at the end-effector is greater than an upper limit of the predetermined range (also referred to herein as the first threshold or the high threshold).

If it is determined at S307 that the current temperature is greater than the high threshold, at S311, the power input to the medical device may be modulated such that the estimated current temperature is reduced to be below the first threshold over a predetermined number of sampling cycles. For example, the power input to the medical device is modulated such that the estimated current temperature is reduced to be below the first threshold over 1, 2, 3, 4, 5, 6, 7, 8, 9, 10, 12, 14, 16, 18, 20, 25, 30 or any number between any two of these numbers, subsequent sampling cycles. Modulation can include any one or more of turning the power input OFF, reducing the power input, pulsing the power input within a range at a certain frequency, or a combination thereof.

In some embodiments, if the estimated current temperature at the end-effector is greater than the first threshold, the power input to the medical device may be turned OFF. In such embodiments, the power may be turned back ON after a certain amount of time or after the estimated current temperature at the end-effector has reduced below the first threshold by a predetermined amount. Thus, in some embodiments, regulating the temperature at the end-effector may include estimating the amount of time for which to keep the power turned OFF based on the rate at which the estimated current temperature at the end-effector decreases after the power has been turned OFF. In some embodiments, the power to the medical device may be turned back ON after the estimated current temperature at the end-effector decreases below the first threshold by a certain amount. In some embodiments, the rate of cooling at the end-effector is predetermined based on parameters such as, for example, the shape and size of the end-effector, the type of the treatment portion, the size and shape of the treatment portion, the contact area between the body tissue and the treatment portion, and/or physical properties (specific heat, thermal conductivity, etc.) of the treatment portion and/or the body tissue in contact with the treatment portion.

In some embodiments, at S313 a warning signal may be provided when the estimated current temperature at the end-effector increases above a first threshold. The warning signal may be provided through audio, visual, audiovisual, and/or haptic feedback mechanism or a combination thereof. In some embodiments, the intensity of the warning signal may be dependent on the amount by which the estimated current temperature exceeds the first threshold. For example, the warning signal may be an audio signal at a first decibel level when the estimated current temperature increases above the first threshold, and the intensity of the audio signal increases to a second decibel level if the estimated current temperature exceeds a second threshold temperature greater than the first threshold temperature.

In some embodiments, the estimated current temperature at the end-effector is displayed on a display. In some embodiments, the estimated current temperature is displayed as a numerical value. In some embodiments, the estimated current temperature is displayed in the form of a graph which may also show an upper and/or a lower limit of a predetermined range of temperatures in which the procedure can be performed safely and effectively. Thus, the clinician may be able to visualize whether the current temperature at the end-effector is within a safe range and take appropriate action if the current temperature at the end-effector is not within the safe range, or is trending to not be within the safe range.

Additionally or optionally, in some embodiments, regulating the temperature at the end-effector may include separating the end-effector from the treatment portion. For example, in embodiments where the end-effector is a grasper used for welding portions of tissue, the tissue may be released from the grasper if the estimated current temperature at the end-effector exceeds the first threshold.

If it is determined that the current temperature is not greater than the high threshold, the method reverts to S309 where the provision of the electrical power to the end-effector is continued.

Although described above with respect to power input to the medical device, the disclosed process can, in addition or alternatively, utilize an amount of electrical power consumed by the medical device, an amount of electrical power applied to the end-effector, an amount of electrical power consumed by the end-effector, or a combination thereof.

Thus, by accurately estimating the current temperature at the end-effector, the temperature at the end-effector may be appropriately regulated to be within a certain temperature range to enable effective and safe performance of the procedure.

Although the present invention has been described in connection with the above exemplary embodiments, it will be appreciated by those skilled in the art that additions, deletions, modifications, and substitutions not specifically described may be made without department from the spirit and scope of the invention as defined in the appended claims.

### C. Further Considerations

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The singular forms “a,” “an,” and “the” include plural referents unless the context clearly dictates otherwise. Thus, for example, reference to “a plunger component” includes reference to one or more plunger components, and reference to “the magnet” includes reference to one or more magnets.

In one or more aspects, the terms “about,” “substantially,” and “approximately” may provide an industry-accepted tolerance for their corresponding terms and/or relativity between items, such as from less than one percent to five percent.

As used herein, the term “substantially” refers to the complete or nearly complete extent or degree of an action, characteristic, property, state, structure, item, or result.

It is to be understood that a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 0.5 to 10 cm” should be interpreted to include not only the explicitly recited values of about 0.5 cm to about 10.0 cm, but also include individual values and sub-ranges within the indicated range. Thus, included in this numerical range are individual values such as 2, 5, and 7, and sub-ranges such as from 2 to 8, 4 to 6, etc. This same principle applies to ranges reciting only one numerical value. Furthermore, such an interpretation should apply regardless of the breadth of the range or the characteristics being described.

Unless otherwise defined, all technical and scientific terms used herein have the same meaning as commonly understood to one of ordinary skill in the art to which this disclosure belongs. Although any methods, devices and materials similar or equivalent to those described herein can be used in the practice or testing of the disclosure, representative methods, devices, and materials are described below.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

The techniques described herein may be implemented in hardware, software, firmware, or any combination thereof, unless specifically described as being implemented in a specific manner. Any features described as modules or components may also be implemented together in an integrated logic device or separately as discrete but interoperable logic devices. If implemented in software, the techniques may be realized at least in part by a non-transitory processor-readable storage medium comprising instructions that, when executed, performs one or more of the methods described above.

The non-transitory processor-readable storage medium may comprise random access memory (RAM) such as synchronous dynamic random access memory (SDRAM), read only memory (ROM), non-volatile random access memory (NVRAM), electrically erasable programmable read-only memory (EEPROM), FLASH memory, other known storage media, and the like. The techniques additionally, or alternatively, may be realized at least in part by a processor-readable communication medium that carries or communicates code in the form of instructions or data structures and that can be accessed, read, and/or executed by a computer or other processor. For example, a carrier wave may be employed to carry computer-readable electronic data such as those used in transmitting and receiving electronic mail or in accessing a network such as the Internet or a local area network (LAN). Many modifications may be made to this configuration without departing from the scope or spirit of the claimed subject matter.

Although the detailed description contains many specifics, these should not be construed as limiting the scope of the subject technology but merely as illustrating different examples and aspects of the subject technology. It should be appreciated that the scope of the subject technology includes some embodiments not discussed in detail above. Various other modifications, changes and variations may be made in the arrangement, operation and details of the method and apparatus of the subject technology disclosed herein without departing from the scope of the present disclosure. Unless otherwise expressed, reference to an element in the singular is not intended to mean “one and only one” unless explicitly stated, but rather is meant to mean “one or more.” In addition, it is not necessary for a device or method to address every problem that is solvable (or possess every advantage that is achievable) by different embodiments of the disclosure in order to be encompassed within the scope of the disclosure. The use herein of “can” and derivatives thereof shall be understood in the sense of “possibly” or “optionally” as opposed to an affirmative capability.

What is claimed is:

1. A method for controlling a temperature at an end-effector of a medical instrument connected with a controller, the method comprising:
estimating, based on a first set of parameters, a residual energy associated with (i) a prior amount of electrical power provided to the medical instrument, (ii) a prior amount of electrical power consumed by the medical instrument, (iii) a prior amount of electrical power applied to the end-effector, (iv) a prior amount of electrical power consumed by the end-effector, or (v) a combination thereof;
determining, based on the first set of parameters, a change in energy of the end-effector based on (i) a current amount of electrical power provided to the medical instrument, (ii) a current amount of electrical power consumed by the medical instrument, (iii) a current amount of electrical power applied to the end-effector, (iv) a current amount of electrical power consumed by the end-effector, or (v) a combination thereof;
estimating a residual temperature of the end-effector associated with the residual energy;
estimating a change in temperature of the end-effector associated with the change in energy;
estimating a current temperature at the end-effector based on the residual temperature of the end-effector associated with the residual energy and the change in temperature of the end-effector associated with the change in energy; and
controlling the electric power provided to the medical instrument to maintain the current temperature at the end-effector within a predetermined range.

2. The method of claim 1, wherein controlling the electric power comprises decreasing the electric power provided to the end-effector when the estimated current temperature is greater than a first threshold temperature.

3. The method of claim 2, wherein controlling the electric power comprises increasing the electric power provided to the end-effector when the estimated current temperature is lower than a second threshold temperature.

4. The method of claim 3, further comprising providing a first warning signal upon a determination that the estimated current temperature is greater than the first threshold temperature.

5. The method of claim 4, further comprising increasing an intensity of the first warning signal if the estimated current temperature continues to increase above the first threshold temperature.

6. The method of claim 5, further comprising:
determining, during a predetermined period of time, a total amount of time for which the current temperature at the end-effector is estimated to be above a first threshold; and
providing a second warning signal if the total amount of time is greater than an allowed threshold.

7. The method of claim 6, further comprising estimating an amount of time for which to stop providing electric power to the medical instrument based on the estimated current temperature, the first threshold temperature and a pre-calculated rate of cooling.

8. A medical device for applying heat to a tissue, the device comprising:
a medical instrument including an end-effector configured to contact the tissue and to transmit heat to the contacted tissue; and
a controller including:
a power source configured to provide electric power to the medical instrument; and
a processor configured to:
compute an amount of electric power converted to heat at the end-effector,
estimate a current temperature at the end-effector based on inputs including: (i) at least one first characteristic, (ii) the amount of electric power converted to heat at the end-effector, and (iii) a time for which the electric power is provided, and
control the electric power provided to the medical instrument to maintain the current temperature at the end-effector within a predetermined range.

9. The medical device of claim 8, wherein the processor is further configured to:
estimate a temperature associated with the residual energy,
estimate a change in temperature associated with the change in energy of the end-effector based on the first set of parameters, and
estimate the current temperature at the end-effector based on the temperature associated with the residual energy and the change in temperature associated with the change in energy.

10. The medical device of claim 9, wherein the first set of parameters includes one or more of a mass of a treatment portion of the end-effector, a shape of the treatment portion of the end-effector, a size of the treatment portion of the end-effector, and a specific heat of the treatment portion of the end-effector.

11. The medical device of claim 10, wherein the processor is configured to decrease the electric power provided to the end-effector when the estimated current temperature is greater than a first threshold temperature.

12. The medical device of claim 11, wherein the processor is further configured to increase the electric power provided to the medical instrument when the estimated current temperature is lower than a second threshold temperature.

13. The medical device of claim 8, wherein the medical instrument includes a display on which the current temperature is displayed.

14. A controller for controlling a medical device, the controller comprising:
a power source configured to provide electric power to a medical instrument of the medical device; and
a processor configured to:
compute an amount of electric power converted to heat at an end-effector of the medical instrument,
estimate a current temperature at the end-effector based on inputs comprising: (i) at least one first characteristic, (ii) the amount of electric power converted to heat at the end-effector, and (iii) a time for which the electric power is provided, and
control the electric power provided to the medical instrument to maintain the current temperature at the end-effector within a predetermined range.

15. The controller of claim 14, wherein the processor is further configured to:
estimate a temperature associated with the residual energy,
estimate a change in temperature associated with the change in energy of the end-effector based on the first set of parameters, and
estimate the current temperature at the end-effector based on the temperature associated with the residual energy and the change in temperature associated with the change in energy.

16. The controller of claim 15, wherein the first set of parameters includes one or more of a mass of the treatment portion of the end-effector, a shape of the end-effector, a size of the treatment portion of the end-effector, and a specific heat of the treatment portion of the end-effector.

17. The controller of claim 16, wherein processor is configured to decrease the electric power provided to the end-effector when the estimated current temperature is greater than a first threshold temperature.

18. The controller of claim 17, wherein the processor is further configured to increase the electric power provided to the medical instrument when the estimated current temperature is lower than a second threshold temperature.
